(19) United States
(12) Patent Application Publication
(10) Pub. No.: US 2010/0215549 A1
(43) Pub. Date: Aug. 26, 2010

(54) CATALYTIC COMBUSTION AROMA DIFFUSER WITH REFILL

(75) Inventor: Alain Corda, Pomponne (FR)

Correspondence Address: ARENT FOX LLP, 1050 CONNECTICUT AVENUE, N.W., SUITE 400, WASHINGTON, DC, 20036, US

(73) Assignee: EUROP COSMETICS, Torcy (FR)

(21) Appl. No.: 12/377,328

(22) PCT Filed: Sep. 19, 2007
(86) PCT No.: PCT/FR2007/001530

§ 371 Date: Sep. 15, 2009

(30) Foreign Application Priority Data
Sep. 19, 2006 (FR) 0608192

(51) Int. Cl. A61L 9/02; A61L 9/03

(52) U.S. Cl. 422/122; 422/310

(57) ABSTRACT

The invention relates to a device for diffusing substance by catalytic combustion allowing the emission of volatile matter comprising: a container (4) containing the liquid to be vaporized; said container (4) having a wick (6) comprising a first end which is dipped in said liquid to be vaporized and a second end which is outside of said container (4); and a diffusion head (2) comprising a catalytic combustion area and a vaporization area; characterized in that said diffusion head (2) is connected detachably to said second end of said wick (6).

The present invention relates to the field of catalytic-combustion diffusers.

The present invention relates more particularly to a device for diffusing a substance by catalytic combustion applying particularly to the diffusion in the air of an aroma or perfume or even insecticide.

The prior art and in particular the patent FR 2 530 144 already describes diffusers provided with a catalytic diffusion head disposed on the spout of a body containing a perfumed fuel. A wick, the first end of which is connected to the diffusion head and the second end of which is immersed in the perfumed fuel contained in the body. The fuel rises by capillarity in the mesh and impregnates the diffusion head formed from a porous material. Ignition is initiated at the diffusion head by an external flame, which ignites the perfumed fuel impregnating the diffusion head. The ignition phase corresponds to the presence of the flame on the diffusion head. Once the flame is extinguished, the functioning of the perfume diffuser is provided by the catalytic combustion generated by the combustion zone of the diffusion head. This is because the catalytic combustion zone of the diffusion head of these diffusers contains a certain quantity of catalyst, for example platinum. The combustion without flame of the perfumed fuel is produced by the catalytic material, and the heat emitted by this catalytic combustion allows the diffusion of the perfume by evaporation on the vaporisation zone of the diffusion head, which is devoid of catalytic material. The combustion causing a reduction in the fuel in the body, it is necessary for an air inlet to be provided in the body in order to avoid negative pressure within it and therefore poor supply of perfumed fuel to the diffusion head. However, the refilling of this device according to the prior art requires removing the diffusion head and the wick to which it is connected in order to fill the reservoir. This manipulation represents a risk of burning for the user since the diffusion head remains hot for a long period even after extinction.

In order to facilitate the filling of the diffusion devices, the prior art and in particular the document EP 1 637 169 describes a device comprising a reservoir provided with two openings. The first opening allows passage of the wick connected to the diffusion head and the second allows filling of the diffuser.

However, when the reservoir of the devices of the prior art is not completely empty, changing aroma constitutes a real problem for the user. This is because drainage of the reservoir prior to any filling with a new perfumed fuel does not suffice to prevent any inconvenience relating to the mixing of the aromas. The wick used being the same, it remains impregnated with the old perfumed fuel and constitutes a site of mixing of the aromas giving rise to the diffusion of the mixture until the previous aroma is diluted.

The devices according to the prior art also have a relatively short life and, over time, have various operating problems relating to the carbonisation of the wick over time.

Moreover, when refilling with a new fuel, the user may use an inappropriate fuel, a source of potential accident or degradation of the diffusion device.

In addition, the filling of the devices of the prior art constitutes per se a risky manipulation since the fuel is flammable and may therefore be a source of fire, explosion or burning by contact. In addition, direct contact of the skin with the chemical products to be vaporised constitutes a potential hazard for the user. Finally, the fact that the perfumed fuel may be situated outside the device for which it is designed, in other words the accessibility of the fuel, represents a risk of accidental ingestion.

The devices according to the prior art are responsible for many fires. This is because simple overturning or simple impact on a functioning diffuser may cause release of the fuel and its ignition by the diffusion head in combustion.

The present invention sets out to remedy these drawbacks of the prior art by proposing a device for diffusing a substance by catalytic combustion allowing the emission of volatile materials, comprising: a reservoir containing the liquid to be vaporised, the said reservoir being provided with a wick comprising a first end immersed in the said liquid to be vaporised and a second end outside the said reservoir, the said device also comprising a diffusion head composed of a catalytic combustion zone and a vaporisation zone; the said diffusion head being connected removably to the said second end of the said wick.

Advantageously, the said wick is kept fixed to the reservoir by an obturation element for the said reservoir so that the wick is in a single piece with the reservoir and the liquid to be vaporised is inaccessible. Thus this assembly presents no risk of burning fire for the user.

In addition, at least the said second end of the said wick is provided with a means of stiffening the wick so that the wick is easily inserted in the diffusion head of the device.

Advantageously, the said reservoir, the said obturation element, the said wick and the said means of stiffening the wick are assembled permanently so as to constitute a refill. Thus, when the device has to be refilled, the user no longer has any direct contact with the fuel but must simply change the refill. This refill allows rapid charging of the device without danger.

According to another embodiment of the invention, the said diffusion head is at least partly held by a housing close to or on the second end of the said wick. Advantageously, the housing is a protective housing for protecting the reservoir from any impact.

Advantageously the said housing comprises a support element removably holding the said diffusion head. The diffusion head can then be changed while keeping the rest of the device. The said support element comprises a recess providing passage for the said wick. This recess affords direct contact between the diffusion head and the top end of the wick when the refill in introduced, thus improving diffusion.

According to a variant of the invention, the said housing comprises a body intended to contain the said refill, the said support element and the said body forming a single-piece assembly. The said body is provided at its bottom part with an opening suitable for allowing the insertion of the said reservoir.

According to another variant, the said body is provided at its top part with an opening suitable for allowing insertion of the said reservoir.

Advantageously, the said diffuser comprises a perforated cover provided with recesses able to be closed off by rotation of an internal perforated element with respect to an external perforated element. The said diffusion head can be provided with an adjustable flame reducer for regulating the intensity of the ignition flame of the said diffusion head.

Moreover, the refill is provided with a stopper covering at least the said second end of the said wick so as in the storage position to prevent the evaporation of the liquid to be vaporised.

The invention will be understood better with the help of the description, given below for purely explanatory purposes, of an embodiment of the invention, with reference to the accompanying figures, in which:

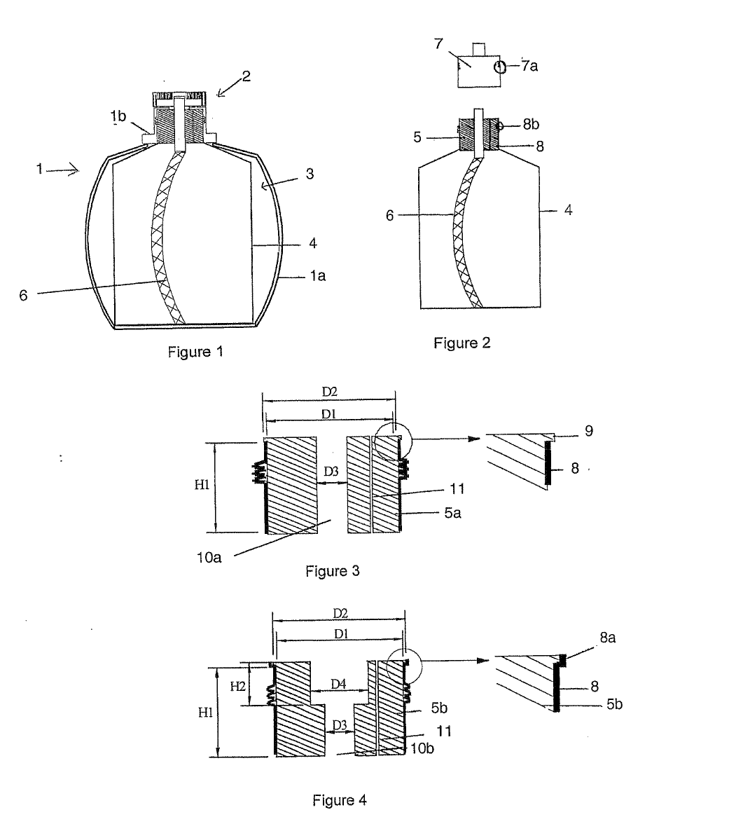

FIG. 1 is a view in vertical section of a first embodiment of the diffusion device;

FIG. 2 is a view in vertical section of a refill of a first embodiment of the diffusion device;

FIG. 3 is a view in vertical section of a first variant of a means of closing off the reservoir of a first embodiment of the diffusion device;

FIG. 4 is a view in vertical section of a second variant of a means of closing off the reservoir of a first embodiment of the diffusion device;

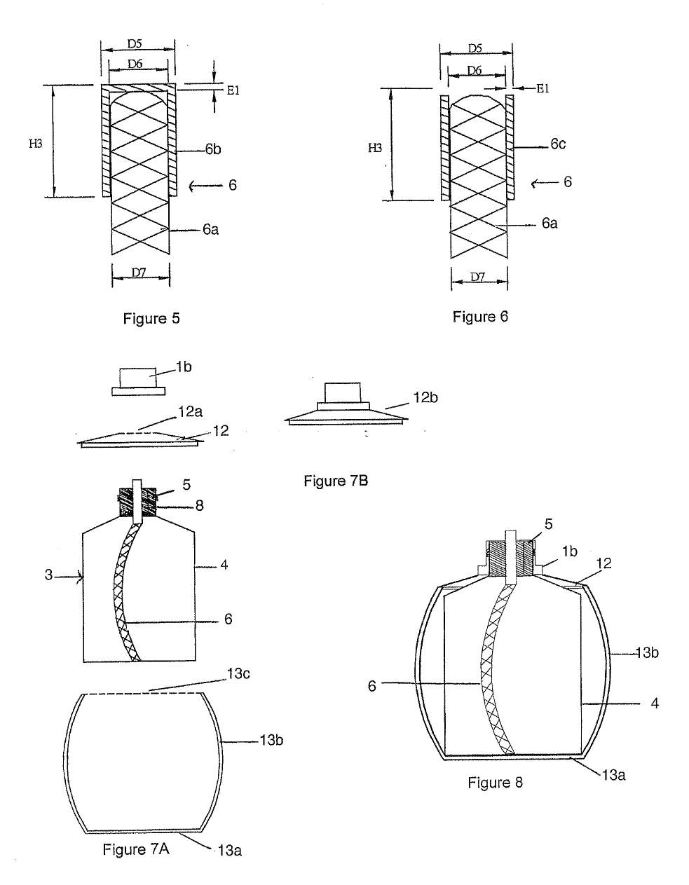

FIG. 5 is a view in vertical section of a first embodiment of the wick of the diffuser according to the invention;

FIG. 6 is a view in vertical section of a second embodiment of the wick of the diffuser according to the invention;

FIG. 7A is an exploded view in vertical section of a first variant of the first embodiment of the diffusion device;

FIG. 7B is a view of a variant of a cover of a first embodiment of the diffusion device;

FIG. 8 is a view in vertical section of a first variant of the first embodiment of the diffusion device;

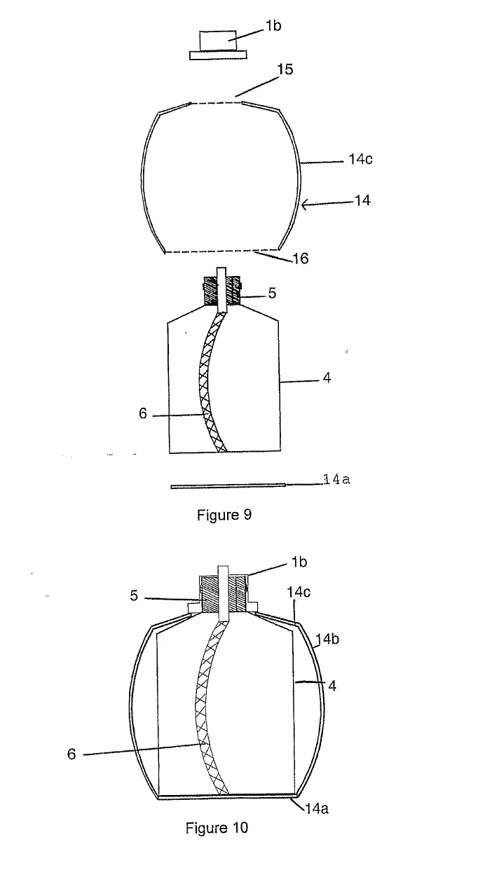

FIG. 9 is an exploded view in vertical section of a second variant of the first embodiment of the diffusion device;

FIG. 10 is a view in vertical section of a second variant of the first embodiment of the diffusion device;

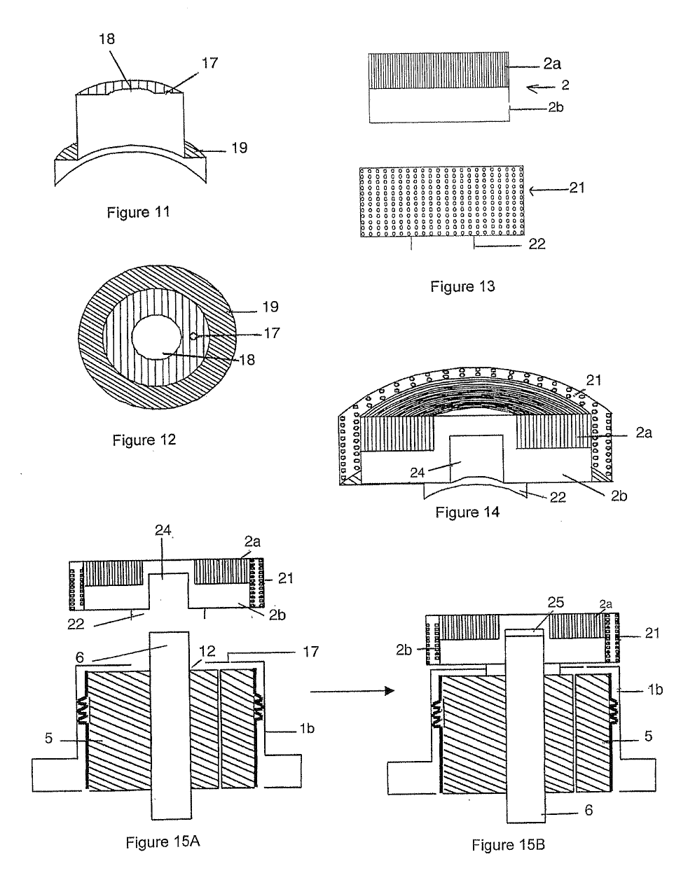

FIG. 11 is a view in vertical section of a support element for the diffusion head of the first embodiment of the diffusion device;

FIG. 12 is a plan view of a support element for the diffusion head of the first embodiment of the diffusion device;

FIG. 13 is an exploded view of the diffusion head provided with a flame reducer of the first embodiment of the diffusion device;

FIG. 14 is a view in vertical section in perspective of the diffusion head provided with a flame reducer of the first embodiment of the diffusion device;

FIG. 15A is an exploded view in Section of the top part of the first embodiment of the diffusion device;

FIG. 15B is a view in section of the top part of the first embodiment of the diffusion device;

FIG. 16 is an exploded front view of the diffuser provided with a cover;

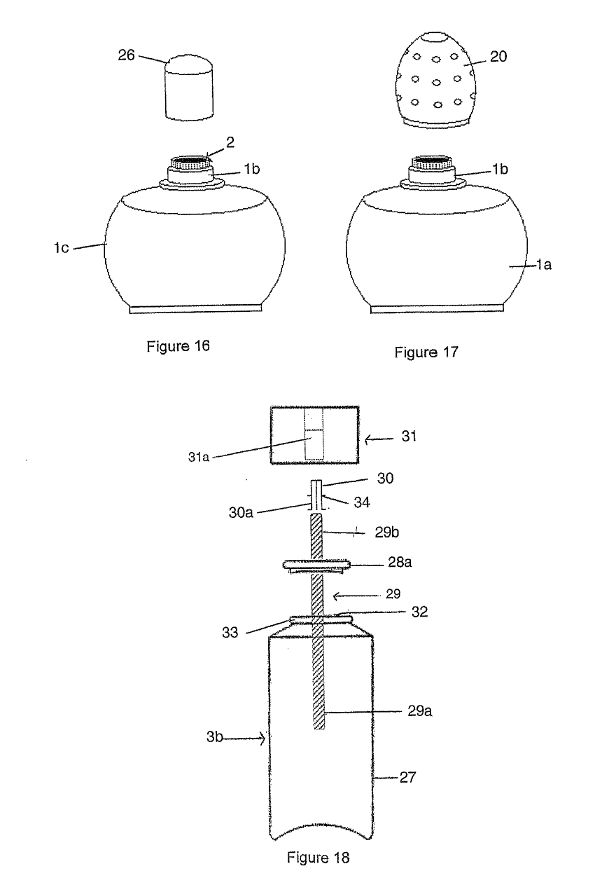

FIG. 17 is an exploded front view of the diffuser provided with a perforated mount;

FIG. 18 is a view in vertical section of a refill of a second embodiment of the diffusion device;

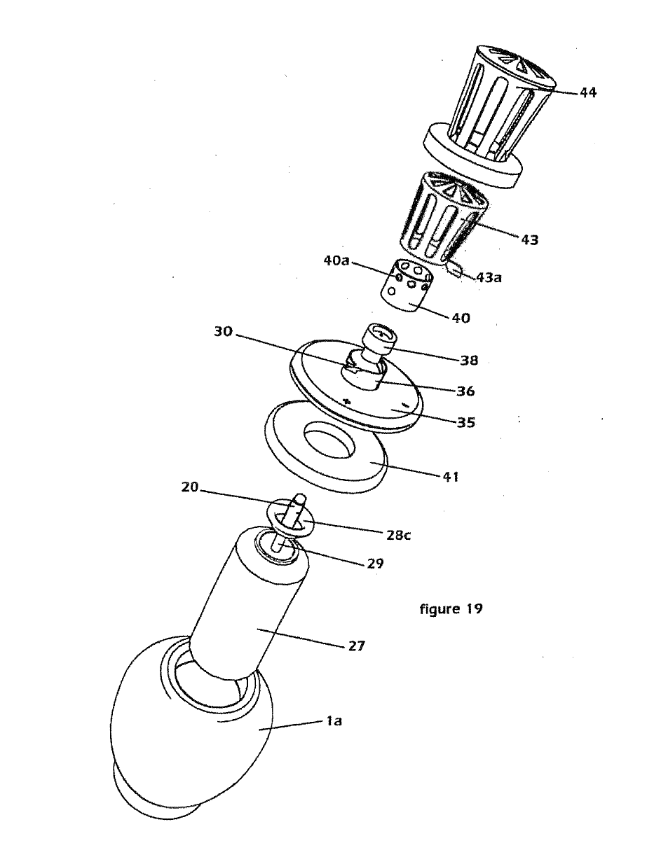

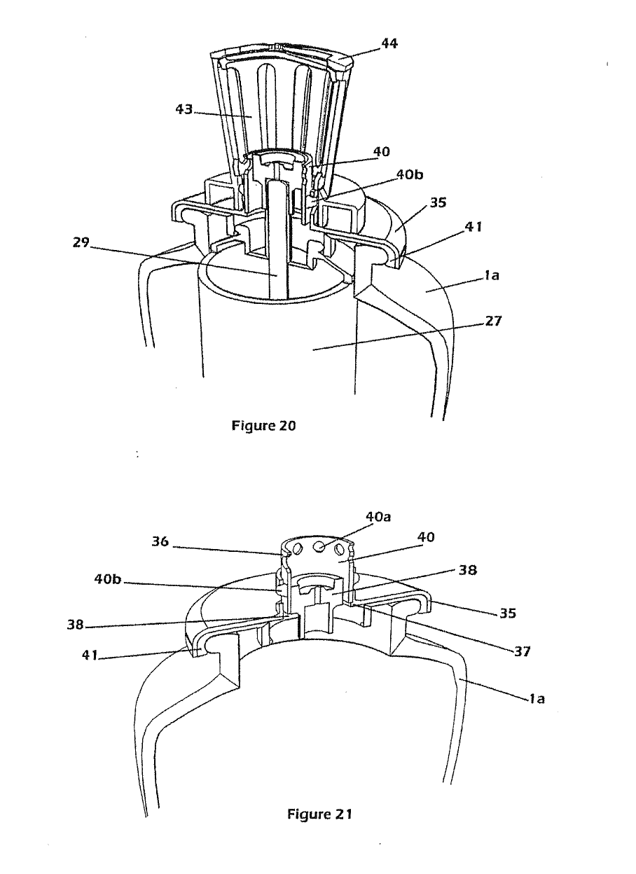

FIG. 19 is an exploded view of a second embodiment of the diffusion device;

FIG. 20 is a view in vertical section of the top part of a second embodiment of the diffusion device;

FIG. 21 is a view in vertical section of the top part of a second embodiment of the diffusion device in the absence of the refill.

Hereinafter, the description of the diffuser is made with reference to its orientation in its vertical idle or operating position with the diffusion head in the top position.

The diffuser according to the invention is composed of a housing 1 for a diffusion head 2 and a refill 3 (see FIG. 1). This housing 1 supports both the diffusion head 2 and the refill 3, holding them in close proximity to each other. The said housing 1 is composed of a body 1a and a support element 1b called a base, which is directly in contact with the diffusion head 2.

According to a first embodiment, the refill 3a (FIG. 2) consists of four elements: a reservoir 4, a means of closing off the reservoir formed by a seal 5, a wick 6 and a storage stopper 7.

The stopper 7 protects the top part of the refill 3a and in particular the wick 6 before any use and in particular during storage of the refill 3a. The stopper 7 is fixed to the spout 8 of the reservoir 4 by screwing or clipping means 7a and 8b.

The reservoir 4 is available in different volumes with different shapes. The material in which the reservoir is designed corresponds to any type of material chemically inert to the perfumed fuels used, and resistant to impacts, thus avoiding any impairment of the seal on the reservoir 4 following a fall of the device or an impact. The material chosen also affords resistance to temperatures of around 60° C., which is the maximum heat transmitted by thermal conduction from the diffusion head 2 to the reservoir 4. The reservoir is preferably composed of PET or aluminium.

The seal 5 has a dual use: it ensures the fluidtightness of the refill 3a by hermetically confining the perfumed fuel in the reservoir 4, and serves as a support element for the wick 6 by keeping it fixed at the spout 8 of the reservoir 4. The seal 5 is inserted at the spout 5, whose shape it adopts. The seal 5 is designed from a material chemically inert to the perfumed fuel used and resistant to flames.

According to a first variant of the seal 5 illustrated in FIG. 3, the seal 5a has a shoulder 9 of diameter D2 with a height of around 1 mm. The shoulder 9 comes into abutment on the spout 8 and thus prevents the seal 5a from being pressed into the reservoir 4 when it is fitted.

The diameter D1 of the seal 5a corresponds to the diameter of the portion of the seal 3a to be inserted in the spout 8. This diameter D1 is determined according to the internal diameter of the spout 8 so that it is force-fitted in the spout 8.

The height H1 of the seal 5a is sufficient to ensure the fluidtightness of the refill 5a and the holding of the wick 6. The seal 5a has two vertical recesses 10a and 11 passing through it. The first recess 10a has a diameter D3a determined with respect to the diameter of the wick 6 passing through the seal 5a, so that the fixing is firm and impervious. The second recess 11 corresponds to the air inlet necessary for the correct functioning of the diffuser. The diameter of the second recess 11 is around 0.5 to 1 mm. In this first embodiment of the seal 5a, the support for the diffusion head 2, namely the base 1b, comes to be positioned at the surface of the seal 5a.

According to a second variant of the seal 5 illustrated in FIG. 4, the seal 5b has a recess 10b broadened at its top part over a height H2 and a diameter D4 so that a base provided with an internal skirt cooperates with the seal 5b in particular by at least partly entering the portion of diameter D4 of the recess 10b. The spout 8 of the reservoir 4 can be provided with a ring 8a covering the shoulder 9 of the seal 5b so as to make it difficult to withdraw the seal 5b once inserted in the spout 8.

The recess 11 of the seal 5, 5a, 5b and the end of the wick 6 constitute fluid communication means between the internal volume of the reservoir and the outside, promoting evaporation of the fuel, when the refill 3a is not used. The fixing of the stopper 7 to the top part of the reservoir makes it possible to obtain total fluidtightness of the refill 3a when it is stored, the stopper 7 being removed in order to be able to fit the refill 3a in the housing 1.

In another variant of the refill 3a, an air inlet in the reservoir can be effected not by a recess 11 in the seal 5, 5a, 5b but by a recess situated in a wall of the reservoir 4 (not illustrated).

The wick 6 of the reservoir 4 according to the invention is fixed to the reservoir 4 so that the assembly consisting of the reservoir 4, the seal 5, 5a, 5b and the wick 6 form a hermetic assembly able to constitute a refill 3a.

The material used for producing the wick 6 has good characteristics of absorption and transfer by capillarity of the perfumed fuel.

The wick 6 forms a rigid structure at least at the portion included in the seal 5, 5a, 5b and its end external to the refill so as to facilitate its fixing by the seal 5, 5a, 5b to the reservoir 4 as well as its fitting at the diffusion head 2 in order to supply it with perfumed fuel.

The embodiment of the wick 6 illustrated in FIG. 5 comprises a textile wick 6a pushed into a means of stiffening the wick 6 formed by this embodiment of a ceramic sleeve 6b. The sleeve 6b of height H3 surrounds the end of the textile wick 6a intended to pass through the seal 5 and to be outside the reservoir 4.

The height of the tube H3 of the sleeve 6b is determined according to the height of the seal 5 or an equivalent able to allow a hermetic closure, but also the emerging height of the tube necessary for the positioning of the diffusion head and the regulation level for supply of perfumed fuel required. The ceramic sleeve 6b surrounding the end of the textile wick 6a situated outside the reservoir 4 allows regulation of the quantity of perfumed fuel migrating by capillarity at the diffusion head 2.

This is because the use of a ceramic sleeve 6b allows the use of a textile wick 6a of reduced diameter D7 and because of this a correspondingly reduced absorption capacity.

The diameter D7 of the textile wick 6a and the inside diameter D6 of the ceramic sleeve 6b are determined according to the desired diffusion and the dimensions of the diffusion head 2 used. The outside diameter D5 of the ceramic sleeve 6b will depend on the thickness E1 and therefore on the material used necessary for good mechanical strength of the sleeve 6b. The outside diameter D5 is in direct relationship with the diameter D3 of the recess 10 in the seal 5, see FIG. 5.

The ceramic used for the ceramic sleeve 6b has sufficient porosity and absorption capacity to transfer the perfumed fuel from the wick 6 to the diffusion head 2, a porous material such as kaolinite, steatite or cordierite.

The means of stiffening the wick 6 may be a ceramic tube 6c illustrated in FIG. 6, the tube 6c is open at both ends so that the transfer of the perfumed fuel to the diffusion head 2 is ensured both by the textile wick 6a and by the ceramic tube 6c. This difference does not have any real repercussions on the functioning of the diffusion head 2 since the ceramic tube 6c serves mainly to position the textile wick 6a in the seal 5 and the diffusion head 2 on the wick 6.

The ceramic tube 6c can be produced with a nonporous material having the required characteristics for meeting the thermal and mechanical stresses. Under these conditions, it is the textile wick 6a that provides the migration of the perfumed fuel to the diffusion head 2, and the limitation of the transfer losses as well as the fitting of the diffusion head 2 on the wick 6 depend on the shape of the ceramic tube 6c used.

The invention allows the use of a wick 6 made entirely from porous ceramic (not illustrated).

The refill 3a is positioned in a housing 1 provided with a diffusion head 2, this housing 1 comprises a decorative body 1a and a base 1b allowing the assembly of the catalysis perfume diffuser. The introduction of the refill 3a into the body 1a can be envisaged according to two modes.

In a first variant of the housing 1 illustrated in FIGS. 7A and 8, the refill 3a is introduced through the top of a body 13. The body 13 comprises a bottom 13a and a lateral wall 13b, the top edge of which delimits an opening 13c intended to allow the introduction of the refill 3a into the body 13. The opening 13c is partly closed off by a cover 12 after the introduction of the refill 3a. The cover 12 has at its centre a recess 12a allowing passage for the spout 8 of the refill 3a. The base 1b of the diffuser comes to rest partly on the spout 8 in order to conceal the latter and partly on the rim of the recess 12a in the cover 12.

The cover 12 can be fitted on the base 1b wall against wall or fixed by any fixing means such as for example screwing or clipping means or lugs.

In order to avoid having a separate lid 12 and base 1b, it is possible to produce these two parts in a single part forming a cover 12b (see FIG. 7B).

The design materials for the cover 12b and base 1b should resist the oxidation related to the perfumed fuel vapours as well as the heat and in particular these two components should resist flames.

The second variant of the housing 1 is illustrated in FIGS. 9 and 10 and makes it possible to introduce the refill 3a through the bottom of the body 14. The body 14 has a lateral wall 14b, the top edge of which delimits a top opening 15. The top free edge of the lateral wall 14b is provided with a radial expansion 14c intended to support the base 1b of the diffuser. The body 14 comprises a bottom opening 16 delineated by the free edges of the lateral wall 14b and intended to allow passage for the refill 3a when it is inserted in the housing 1.

A removable base plate 14a is provided in order to close off the opening 16 in the body 14 after the insertion of the refill 3a. The base plate 14a can be fitted on the body 14, wall against wall or fixed by any fixing means such as for example screwing or clipping means or lugs.

The top opening 15 allows passage for the spout 8 of the refill 3a. The base 1b rests partly on the spout 8 and partly on the free edge of the radial expansion 14c of the body 14.

The choice of the design materials of the body 1a, 13a and 14 of the diffuser is very wide because there is no contact with the perfumed fuel and it suffers only very slight thermal stresses.

The base 1b illustrated in FIGS. 11 and 12 has on its top face a very fine first hole 17 approximately 1 mm in diameter intended to be positioned facing the recess 11 in the seal 5 ensuring fluid communication between the internal volume of the reservoir 4 and the outside atmosphere. This is because, in the absence of this hole 17, the walls of the base 1b would close off the recess 11 in the seal 5. The second opening 18 allows passage for the wick 6 through the base 1b and the fixing of the diffusion head 2. In the absence of a recess 11 in the seal 5, 5a or 5b, the hole 17 proves unnecessary.

The shoulder 19, provided on the base 1b, serves both for masking the opening in the body 1a, 13a or 14 of the diffuser and for fixing a perforated mount 20 (see FIG. 17). The addition of a perforated mount 20 on the base 1b makes it possible, in the event of overturning of the diffuser, to prevent any contact of the hot diffusion head with external materials sensitive to heat.

The air inlet hole can be provided in other parts of the base 1b or of the body 1a, 13a or 14 of the diffuser, depending on its position on the refill 3a. The base 1b can be opened entirely on its top part leaving visible the top surface of the spout 8 of the refill 3a.

Once the device is mounted in accordance with the above description, it suffices to position a diffusion head 2 on the wick 6.

To prevent the release of smoke, it suffices to arrange the diffusion head 2 so that, once fitted, it does not come into contact with the wick 6. The heat of the diffusion head 2 transmitted by the heating system suffices for the evaporation of the perfumed fuel from the wick 6 to the diffusion head 2. The perfumed fuel vapours coming into contact with the hot catalytic material are consumed and initiate the catalytic combustion.

A flame reducer 21 can be provided on the diffusion head 2, allowing control of the ignition flame (see FIGS. 13a and 14). The flame reducer 21 constitutes an independent structure that can be fixed to the diffusion head 2 or form a single-piece assembly with the diffusion head 2.

The flame reducer 21 surrounds the diffusion head 2 and is composed of a tube pierced with a multitude of holes 6 (see FIGS. 13a and 14). The presence of holes 6 acts on the ignition flame by controlling the air inlet to the diffusion head 2 while allowing good combustion and diffusion of the perfumed fuel over all the areas of the diffusion head 2.

A flame reducer in the form of a very fine grill or non-pierced tube (not illustrated) can be envisaged on this type of diffuser.

The diameter and height of the flame reducer 21 are elements for controlling the ignition flame. Thus, according to the dimensions of the flame-reducer 21 chosen and the presence or not of holes, the characteristics of the ignition flame and perfume diffusion will be different.

The diffusion head 2 is composed of a combustion zone 2a comprising a precious metal such as platinum providing the catalytic combustion, and a vaporisation zone 2b devoid of precious metal (FIGS. 13a and 14).

The flame reducer 21 is provided with a bottom skirt 22 intended to fit in the opening 18 of the base 1b, so that the base 1b supports the flame reducer 21 and the diffusion head 2 associated with it.

The space 23a present between the seal 5 and the base 1b affords both a sufficient depth of fitting for holding the flame reducer 21 and protection of the seal 5 against the thermal conduction of the material since air is a good thermal insulator (see FIGS. 15a and 15b). The diffusion head 2 has an internal cavity 24 that allows fitting of the top end of the wick 6 inside it. A space 25 can be provided between the top end of the wick 6 and the diffusion head 2 after the placing of the diffusion head 2 on the top end of the wick 6. The presence of air in this space 25 prevents premature wear on the wick 6 by carbonisation, limits the diffusion of liquid perfumed fuel to the diffusion head 2 and promotes the diffusion of perfumed fuel by evaporation. This is because the level of penetration of wick 6 in the diffusion head 2 is a factor that makes it possible to control the diffusion of perfumed fuel.

The diffuser is provided with a stopper 26 and a perforated mount 21 able to fit in the base 1b and be supported in particular by the shoulder 19 of the base 1b (see FIGS. 16 and 17). The stopper 26 (see FIG. 16) makes it possible to stop the combustion at the diffusion head 2 when it is fixed to the diffuser by depriving it of any oxygen.

In order to insert the refill 3a in the housing 1, the stopper 7 storing the refill 3a must be removed. Thus, between the diffusion phases, the fuel contained in the refill 3a may evaporate through the wick 6 and the recess 9 in the seal 5. The evaporation of the fuel between the diffusion phases can be prevented by fitting the stopper 26 of the diffuser providing sealed closure of the refill 3a when the latter is inserted in the housing 1.

The perforated mount 20 (see FIG. 17) is a decorative element that is fixed to the base 1b and protects the diffusion head 2 when the diffuser is used. By coming to be fixed on the base 1b, the perforated mount 20 forms an additional safety element isolating the hot diffusion head 2 from the materials external to the diffusion device.

The refill 3b according to a second embodiment of the diffuser (see FIG. 18) is composed of a reservoir 27, a means of closing off the reservoir formed by a valve cup 28, a textile wick 29, a means of stiffening the wick 29 and a storage stopper 31.

The reservoir 27 consists of a material resistant to impact from heat and is also inert to the perfumed fuel. Composed of a base wall 27a and a lateral wall 27b, the reservoir 27 is provided with an opening 32 bordered by a neck 33 formed on the free edge of the lateral wall 27b.

The valve cup 28 is provided with a transverse collar 28a intended to allow the crimping of the valve cup 28 at the opening 32 so as to close off the reservoir 27. The valve cup 28 has at its centre a recess 28b provided with a ring 28c, the recess 28b being intended to allow passage for the said wick 29.

The wick 29 is formed from a textile material, preferably cotton. The first end 29a of the wick 29 is situated in the reservoir 27 and is immersed in the fuel, the second end 29b is external to the reservoir. The wick 29 passes through the recess 28b in the valve cup 28 on which it is held.

So that the second end 29b of the textile wick 29 is sufficiently rigid to be held close or be included at the diffusion head of the device, it is gripped in a stiffening means which is, in this embodiment, a wick mount 30.

The wick mount 30 is composed of a material extremely resistant to heat, preferably a metal material. With a perforated structure, the wick mount 30 is provided with two series of lugs 34. Each arm 30a of the wick mount 30 being provided with two lugs 34 intended to snap on the top and bottom ends of the ring 28c of the valve cup 28, so as to keep the wick 29 fixed on the valve cup 28 itself crimped on the reservoir 27. Because of this, the fuel in the refill 3b is inaccessible and the refill 3b cannot be opened.

The stopper 31 of the refill 3b fits on the wick mount 30 by means of an internal skirt 31a that affords the hermetic isolation of the wick 29 so as to prevent any evaporation of fuel during storage of the refill 3b.

The refill 3b is inserted in the housing 1 after removal of the storage stopper 31.

The housing 1 is composed of a body 1a in which the reservoir 27 of the refill 3b is inserted. The body 1a is provided with a cover 35 that has at its central part a recess bordered by a peripheral ring 36. The peripheral ring 36 acts as a support element for the diffusion head 38. This is because the diffusion head 38 is inserted in the peripheral ring 36, which prevents any lateral movement. In the presence of the refill 3b, the bottom portion of the diffusion head 38 rests on the top end of the wick 29 of the refill 3b. In a variant, the diffusion head 38 rests on the free edge of the ring 28c of the cover of the refill 3b (not illustrated).

A variant of the cover 35 comprises a bottom extension 37 for the ring 36 provided at its free end with a shoulder 39 for supporting the diffusion head in the absence of the refill 3b in the device (see FIG. 21).

The ring 36 is hollowed out with two spiral grooves 39 extending from the free edge of the ring 36 to its base. Between the diffusion head and the ring 36 of the cover 35, the flame reducer 40 slides. The flame reducer 40 is provided with a hole 40a and two lugs 40b intended to slide along the grooves 39 so that the rotation of the flame reducer 40 causes a vertical translation thereof.

Thus, by regulating the height of the flame reducer 40 according to the diffusion head 38, the user can reduce the intensity of the ignition flame.

A seal 41 can be provided between the cover 35 and the body 1a in order to immobilise the refill 3b and to guarantee a good fixing of the cover 35 on the body 1a.

The device is also provided with a cover 42 serving both as a cover for stopping combustion and a perforated mount protecting the user from any contact with the diffusion head 38.

The cover 42 slides along the ring 36, on which it is held by a fixing means such as clipping. The cover 42 is composed of two perforated elements 43 and 44 inserted one in the other. The internal perforated element 43 comprises a tongue 43a enabling it to be driven in rotation with respect to the external perforated element 44 so as to offset the recesses in each perforated element 43 and 44. By obstructing the recesses in the cover 42, the user can prevent any air inlet and thus stop the combustion at the diffusion head 38.

The invention is described above by way of example. A person skilled in the art is in a position to implement different variants of the invention without for all that departing from the scope of the patent.

1. A device for diffusing a substance by catalytic combustion allowing the emission of volatile materials, comprising:
a reservoir (4, 27) containing a liquid to be vaporised, the said reservoir being provided with a wick (6, 29) comprising a first end immersed in the liquid to be vaporised and a second end outside the reservoir (4, 27),
a diffusion head (2) comprising a catalytic combustion zone and a vaporisation zone,
wherein the diffusion head (2) is removably connected to the second end of the wick.

2. The device according to claim 1, wherein the wick (6, 29) is fixed to the reservoir by a closure element for the reservoir (4, 27).

3. The device according to claim 1 or claim 2, wherein the reservoir (4, 27), the closure element and the wick (6, 29) are assembled permanently so as to constitute a hermetic refill (3a, 3b).

4. The device according to claim 3, the wherein the diffusion head (2) is at least partly kept close to or on the second end of the wick (6, 29) by a protective housing (1), the housing comprising a body (1a) containing the refill (3a, 3b).

5. The device according to claim 4, wherein the housing (1) comprises a support element (1b) removably holding the diffusion head (2).

6. The device according to claim 5, wherein the support element (1b) comprise a recess (10a) giving passage to the wick (6, 29).

7. The device according to claim 5 wherein the support element (1b) and the body (1a) form a single-piece assembly.

8. The device according to claim 4, wherein the body (1a) is provided at a bottom part with an opening (16) suitable for allowing the insertion of the reservoir (4, 27).

9. The device according to claim 4, wherein the body (1a) is provided at a top part with an opening (13c) suitable for allowing the insertion of the reservoir (4, 27).

10. The device according to claim 1, wherein at least the second end of the wick (6, 29) is provided with a means for stiffening the wick (5, 29).

11. The device according to claim 1, wherein the diffuser comprises a perforated cover (12) provided with recesses (12a) suitable for being closed off by rotating an internal perforated element with respect to an external perforated element.

12. The device according to claim 1, wherein the diffusion head (2) is provided with an adjustable flame reducer (21, 40) for regulating the intensity of the ignition flame of the diffusion head.

13. A refill (3a, 3b) for a catalytic combustion diffuser according to claim 1.

14. The refill (3a, 3b) according to claim 13, provided with a stopper (16, 31) fitting on top of at least the second end of the wick (6, 29) so as to avoid, in a storage position, evaporation of the liquid to be vaporised.
